(19) United States
(12) Patent Application Publication
(10) Pub. No.: US 2010/0152198 A1
(43) Pub. Date: Jun. 17, 2010

(54) USE OF C-SRC INHIBITORS IN COMBINATION WITH A PYRIMIDYLAMINOBENZAMIDE COMPOUND FOR THE TREATMENT OF LEUKEMIA

(76) Inventor: Paul W. Manley, Arlesheim (CH)

Correspondence Address: DILWORTH & BARRESE, LLP, 1000 WOODBURY ROAD, SUITE 405, WOODBURY, NY, 11797, US

(21) Appl. No.: 12/295,832

(22) PCT Filed: Apr. 5, 2007
(86) PCT No.: PCT/EP07/53399

§ 371 Date: Oct. 2, 2008

Related U.S. Application Data
(60) Provisional application No. 60/790,473, filed on Apr. 7, 2006.

(51) Int. Cl. A61K 31/506; A61K 31/519; A61K 31/496; A61P 35/02; C07D 487/04

(52) U.S. Cl. 514/252.19; 514/265.1; 514/262.1; 514/253.06; 544/280; 544/262

(57) ABSTRACT

The invention relates to a combination which comprises (a) at least one compound decreasing the c-Src activity and (b) a pyrimidylaminobenzamide compound; to pharmaceutical compositions comprising said combinations; and to a method of treating a warm-blooded animal having leukemia, especially chronic myelogenous leukemia, comprising administering to the animal at least one compound inhibiting the activity of a member of the Src kinase family, the Btk kinase family, the Tec kinase family or a Raf kinase inhibitor, in particular inhibiting the c-Src protein tyrosine kinase activity or inhibiting simultaneously the c-Src protein tyrosine kinase activity and the Bcr-Abl tyrosine kinase activity, in combination with a pyrimidylaminobenzamide compound, in particular 4-Methyl-3-[[4-(3-pyridinyl)-2-pyrimidinyl]amino]-N-[5-(4-methyl-1H-imidazol-1-yl)-3-(trifluoromethyl)phenyl]benzamide.

The invention relates to a method of treating a warm-blooded animal, especially a human, having leukemia comprising administering to the animal (a) at least one compound inhibiting the c-Src protein tyrosine kinase activity and (b) pyrimidylaminobenzamide compound in a quantity which is jointly therapeutically effective against leukemia; a combination which comprises (a) at least one compound decreasing the c-Src activity and (b) a pyrimidylaminobenzamide compound and optionally at least one pharmaceutically acceptable carrier for simultaneous, separate or sequential use; a pharmaceutical composition comprising such a combination; the use of a compound inhibiting the c-Src protein tyrosine kinase activity or the use of the combination of (a) and (b) for the preparation of a medicament for the delay of progression or treatment of leukemia; and to a commercial package or product comprising such a combination of (a) and (b) together with instructions for use thereof in the treatment of leukemia.

Protein tyrosine kinases catalyze the phosphorylation of specific tyrosine residues. One member of this class of enzymes is the c-Src protein tyrosine kinase. Surprisingly, it has now been found that (a) compounds inhibiting the c-Src protein tyrosine kinase activity, especially the compounds described hereinafter, in combination with (b) a pyrimidylaminobenzamide compound are effective against leukemia. Furthermore, it was surprisingly found that the effect in treating leukemia of a combination which comprises (a) at least one compound decreasing the c-Src activity and (b) a pyrimidylaminobenzamide compound is greater than the effects that can be achieved with either type of combination partner alone, i.e. greater than the effects of a monotherapy using only one of the combination partners (a) and (b) as defined herein.

Hence, in a first embodiment, the present invention relates to a combination which comprises (a) at least one compound decreasing the c-Src activity and (b) a pyrimidylaminobenzamide compound, wherein the active ingredients are present in each case in free form or in the form of a pharmaceutically acceptable salt, and optionally at least one pharmaceutically acceptable carrier; for simultaneous, separate or sequential use.

In a broader sense, the present invention relates to a method of treating a warm-blooded animal having leukemia, in particular comprising administering to the animal at least one compound inhibiting the activity of a member of the Src kinase family, in particular src, yes, hck, fyn, lyn, Ick, blk, fgr or Yrk, the activity of a member of the Btk or Tec kinase family or a Raf kinase inhibitor, e.g. BAY 43-9006, in a quantity which is therapeutically effective against leukemia in combination with a Bcr-Abl inhibitor, in particular a pyrimidylaminobenzamide compound such as 4-methyl-3-[[4-(3-pyridinyl)-2-pyrimidinyl]amino]-N-[5-(4-methyl-1H-imidazol-1-yl)-3-(trifluoromethyl)phenyl]benzamide.

The term leukemia as used herein includes, but is not limited to, chronic myelogenous leukemia (CML) and acute lymphocyte leukemia (ALL), especially Philadelphia-chromosome positive acute lymphocyte leukemia (Ph+ALL). Preferably, the variant of leukemia to be treated by the methods disclosed herein is CML.

The term “method of treatment” as used herein includes a treatment effecting the delay of progression of leukemia. The term “delay of progression” as used herein means in particular the administration of a medicament to patients being in a pre-stage or in an early phase of leukemia, in which patients, for example, a pre-form or an early form of leukemia is diagnosed or which patients are in a condition, e.g. a condition resulting from an accident, under which it is likely that a corresponding disease will develop.

The term “compounds inhibiting the c-Src protein tyrosine kinase activity” as used herein means such compounds having an IC₅₀ in the range of 1 to 3000 nM, preferably in the range of 1 to 500 nM, in the proliferation test using bcr-Abl transfected 32D cells described hereinafter. The term includes, but is not limited to, compounds belonging to the structure classes of pyrrolopyrimidines, especially pyrrolo[2,3-d]pyrimidines, purines, pyrazo-pyrimidines, especially pyrazo[3,4-d]pyrimidines, pyrazopyrimidines, especially pyrazo[3,4-d]pyrimidines and pyridopyrimidines, especially pyrido[2,3-d]pyrimidines. Preferably, the term relates to those compounds disclosed in WO 96/10028, WO 97/28161, WO97/32879 and WO97/49706 and, more preferably, to the single compounds of formulae I to VIII, most preferably to the compound of formula I and V, in particular the compound of formula I.

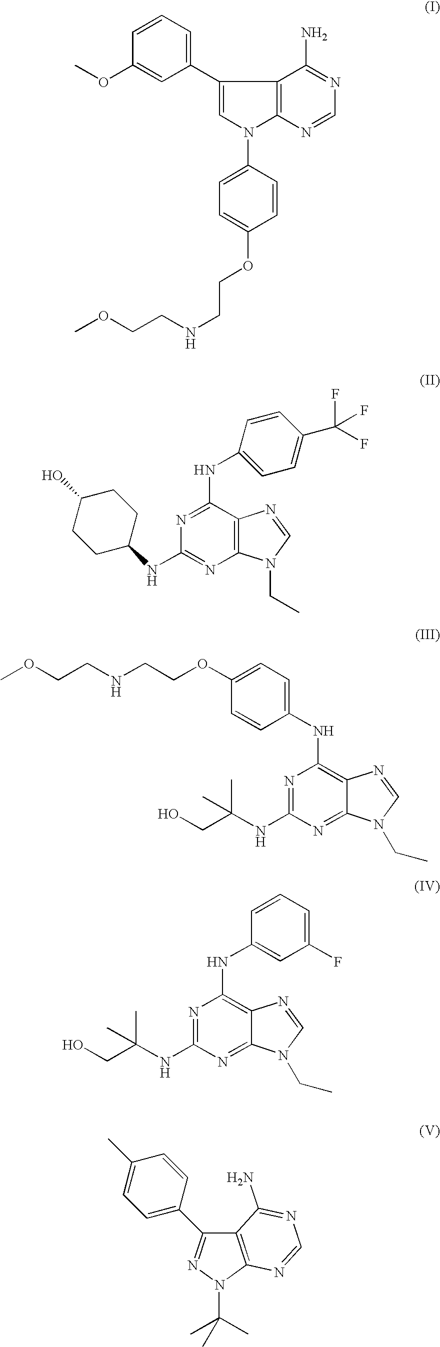

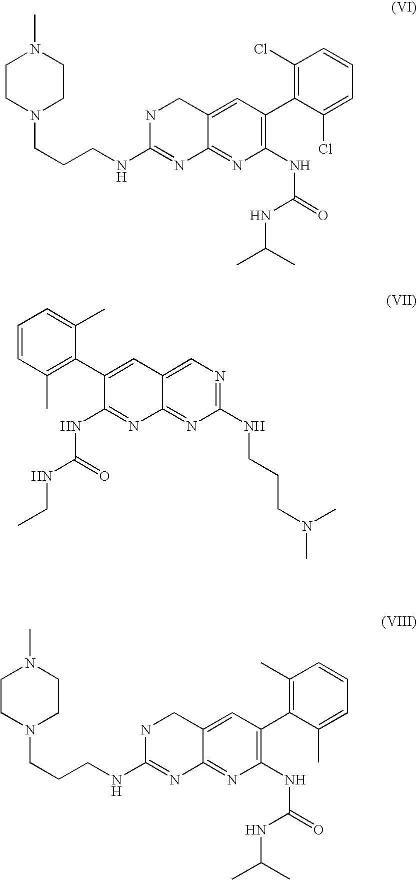

The compounds which are generically and specifically disclosed in WO 96/10028, WO 97/28161, WO97/32879 and WO97/49706, in each case in particular in the compound claims and the final products of the working examples, are hereby incorporated into the present application by reference to these publications. The compounds can be prepared and administered as described in the cited documents, respectively. The compound of formula I can be prepared and formulated as described in WO 96/10028. The compound of formula II and its preparation is disclosed in Example 111c3 of WO 97/16452. The compound of formula IV can be prepared in analogy thereof. Both latter compounds can be formulated as described in WO 97/16452. The compound of formula III is discussed by R. Gamse et al. in J. Bone Miner. Res. 14 (Suppl. 1), 1999, S487. The compound of formula V is also known as PP1. The preparation of PP1 is described by T. Schindler, F. Sicheri et al in Molecular Cell, 1999 (3), 639, 647. The compounds of formula VI, VII and VIII are described in the following documents and the literature cited therein: J. M. Hamby et al, J. Med. Chem. 40, 1997, 2296-2303; R. L. Panek et al, J. Pharmacol. Exp. Ther. 283, 1997, 1433-1444; and S. R. Klutchko et al, J. Med. Chem. 41, 1998, 3276-3292.

Other src inhibitors include SKI606, also known as bosutinib, from Wyeth, and the compound dasatinib, also know as Spyrcel from Bristol-Myers Squibb, which is disclosed in WO 00/62778 and U.S. Pat. No. 6,596,746.

The present invention relates to pyrimidylaminobenzamide compounds of formula IX:

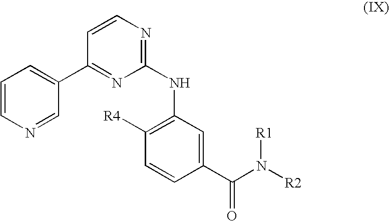

wherein

R₁ represents hydrogen, lower alkyl, lower alkoxy-lower alkyl, acyloxy-lower alkyl, carboxy-lower alkyl, lower alkoxycarbonyl-lower alkyl, or phenyl-lower alkyl;

R₂ represents hydrogen, lower alkyl, optionally substituted by one or more identical or different radicals R₃, cycloalkyl, benzcycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted;

and R₃ represents hydroxy, lower alkoxy, acyloxy, carboxy, lower alkoxycarbonyl, carbamoyl, N-mono- or N,N-disubstituted carbamoyl, amino, mono- or disubstituted amino, cycloalkyl, heterocyclyl, an aryl group, or a mono- or bicyclic heteroaryl group comprising zero, one, two or three ring nitrogen atoms and zero or one oxygen atom and zero or one sulfur atom, which groups in each case are unsubstituted or mono- or polysubstituted;

or wherein R₁ and R₂ together represent alkylene with four, five or six carbon atoms optionally mono- or disubstituted by lower alkyl, cycloalkyl, heterocyclyl, phenyl, hydroxy, lower alkoxy, amino, mono- or disubstituted amino, oxo, pyridyl, pyrazinyl or pyrimidinyl; benzalkylene with four or five carbon atoms; oxaalkylene with one oxygen and three or four carbon atoms; or azaalkylene with one nitrogen and three or four carbon atoms wherein nitrogen is unsubstituted or substituted by lower alkyl, phenyl-lower alkyl, lower alkoxycarbonyl-lower alkyl, carboxy-lower alkyl, carbamoyl-lower alkyl, N-mono- or N,N-disubstituted carbamoyl-lower alkyl, cycloalkyl, lower alkoxycarbonyl, carboxy, phenyl, substituted phenyl, pyridinyl, pyrimidinyl, or pyrazinyl;

R₄ represents hydrogen, lower alkyl, or halogen;

and a N-oxide or a pharmaceutically acceptable salt of such a compound for the preparation of a pharmaceutical composition for the treatment of kinase dependent diseases.

The general terms used hereinbefore and hereinafter preferably have within the context of this disclosure the following meanings, unless otherwise indicated:

The prefix “lower” denotes a radical having up to and including a maximum of 7, especially up to and including a maximum of 4 carbon atoms, the radicals in question being either linear or branched with single or multiple branching.

Where the plural form is used for compounds, salts, and the like, this is taken to mean also a single compound, salt, or the like.

Any asymmetric carbon atoms may be present in the (R)-, (S)- or (R,S)-configuration, preferably in the (R)- or (S)-configuration. The compounds may thus be present as mixtures of isomers or as pure isomers, preferably as enantiomer-pure diastereomers.

The invention relates also to possible tautomers of the compounds of formula IX.

Lower alkyl is preferably alkyl with from and including 1 up to and including 7, preferably from and including 1 to and including 4, and is linear or branched; preferably, lower alkyl is butyl, such as n-butyl, sec-butyl, isobutyl, tert-butyl, propyl, such as n-propyl or isopropyl, ethyl or methyl. Preferably lower alkyl is methyl, propyl or tert-butyl.

Lower acyl is preferably formyl or lower alkylcarbonyl, in particular acetyl.

An aryl group is an aromatic radical which is bound to the molecule via a bond located at an aromatic ring carbon atom of the radical. In a preferred embodiment, aryl is an aromatic radical having 6 to 14 carbon atoms, especially phenyl, naphthyl, tetrahydronaphthyl, fluorenyl or phenanthrenyl, and is unsubstituted or substituted by one or more, preferably up to three, especially one or two substituents, especially selected from amino, mono- or disubstituted amino, halogen, lower alkyl, substituted lower alkyl, lower alkenyl, lower alkynyl, phenyl, hydroxy, etherified or esterified hydroxy, nitro, cyano, carboxy, esterified carboxy, alkanoyl, benzoyl, carbamoyl, N-mono- or N,N-disubstituted carbamoyl, amidino, guanidino, ureido, mercapto, sulfo, lower alkylthio, phenylthio, phenyl-lower alkylthio, lower alkylphenylthio, lower alkylsulfinyl, phenylsulfinyl, phenyl-lower alkylsulfinyl, lower alkylphenylsulfinyl, lower alkylsulfonyl, phenylsulfonyl, phenyl-lower alkylsulfonyl, lower alkylphenylsulfonyl, halogen-lower alkylmercapto, halogen-lower alkylsulfonyl, such as especially trifluoromethanesulfonyl, dihydroxybora (—B(OH)₂), heterocyclyl, a mono- or bicyclic heteroaryl group and lower alkylene dioxy bound at adjacent C-atoms of the ring, such as methylene dioxy. Aryl is more preferably phenyl, naphthyl or tetrahydronaphthyl, which in each case is either unsubstituted or independently substituted by one or two substituents selected from the group comprising halogen, especially fluorine, chlorine, or bromine; hydroxy; hydroxy etherified by lower alkyl, e.g. by methyl, by halogen-lower alkyl, e.g. trifluoromethyl, or by phenyl; lower alkylene dioxy bound to two adjacent C-atoms, e.g. methylenedioxy, lower alkyl, e.g. methyl or propyl; halogen-lower alkyl, e.g. trifluoromethyl; hydroxy-lower alkyl, e.g. hydroxymethyl or 2-hydroxy-2-propyl; lower alkoxy-lower alkyl; e.g. methoxymethyl or 2-methoxyethyl; lower alkoxycarbonyl-lower alkyl, e.g. methoxy-carbonylmethyl; lower alkynyl, such as 1-propynyl; esterified carboxy, especially lower alkoxycarbonyl, e.g. methoxycarbonyl, n-propoxy carbonyl or iso-propoxy carbonyl; N-mono-substituted carbamoyl, in particular carbamoyl monosubstituted by lower alkyl, e.g. methyl, n-propyl or iso-propyl; amino; lower alkylamino, e.g. methylamino; di-lower alkylamino, e.g. dimethylamino or diethylamino; lower alkylene-amino, e.g. pyrrolidino or piperidino; lower oxaalkylene-amino, e.g. morpholino, lower azaalkylene-amino, e.g. piperazino, acylamino, e.g. acetylamino or benzoylamino; lower alkylsulfonyl, e.g. methylsulfonyl; sulfamoyl; or phenylsulfonyl.

A cycloalkyl group is preferably cyclopropyl, cyclopentyl, cyclohexyl or cycloheptyl, and may be unsubstituted or substituted by one or more, especially one or two, substituents selected from the group defined above as substituents for aryl, most preferably by lower alkyl, such as methyl, lower alkoxy, such as methoxy or ethoxy, or hydroxy, and further by oxo or fused to a benzo ring, such as in benzcyclopentyl or benzcyclohexyl.

Substituted alkyl is alkyl as last defined, especially lower alkyl, preferably methyl; where one or more, especially up to three, substituents may be present, primarily from the group selected from halogen, especially fluorine, amino, N-lower alkylamino, N,N-di-lower alkylamino, N-lower alkanoylamino, hydroxy, cyano, carboxy, lower alkoxycarbonyl, and phenyl-lower alkoxycarbonyl. Trifluoromethyl is especially preferred.

Mono- or disubstituted amino is especially amino substituted by one or two radicals selected independently of one another from lower alkyl, such as methyl; hydroxy-lower alkyl, such as 2-hydroxyethyl; lower alkoxy lower alkyl, such as methoxy ethyl; phenyl-lower alkyl, such as benzyl or 2-phenylethyl; lower alkanoyl, such as acetyl; benzoyl; substituted benzoyl, wherein the phenyl radical is especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl, and carbamoyl; and phenyl-lower alkoxycarbonyl, wherein the phenyl radical is unsubstituted or especially substituted by one or more, preferably one or two, substituents selected from nitro, amino, halogen, N-lower alkylamino, N,N-di-lower alkylamino, hydroxy, cyano, carboxy, lower alkoxycarbonyl, lower alkanoyl, and carbamoyl; and is preferably N-lower alkylamino, such as N-methylamino, hydroxy-lower alkylamino, such as 2-hydroxyethylamino or 2-hydroxypropyl, lower alkoxy lower alkyl, such as methoxy ethyl, phenyl-lower alkylamino, such as benzylamino, N,N-di-lower alkylamino, N-phenyl-lower alkyl-N-lower alkylamino, N,N-di-lower alkylphenylamino, lower alkanoylamino, such as acetylamino, or a substituent selected from the group comprising benzoylamino and phenyl-lower alkoxycarbonylamino, wherein the phenyl radical in each case is unsubstituted or especially substituted by nitro or amino, or also by halogen, amino, N-lower alkylamino, N,N-di-lower alkylamino, hydroxy, cyano, carboxy, lower alkoxycarbonyl, lower alkanoyl, carbamoyl or aminocarbonylamino. Disubstituted amino is also lower alkylene-amino, e.g. pyrrolidino, 2-oxopyrrolidino or piperidino; lower oxaalkylene-amino, e.g. morpholino, or lower azaalkylene-amino, e.g. piperazino or N-substituted piperazino, such as N-methylpiperazino or N-methoxycarbonylpiperazino.

Halogen is especially fluorine, chlorine, bromine, or iodine, especially fluorine, chlorine, or bromine.

Etherified hydroxy is especially C₈-C₂₀alkyloxy, such as n-decyloxy, lower alkoxy (preferred), such as methoxy, ethoxy, isopropyloxy, or tert-butyloxy, phenyl-lower alkoxy, such as benzyloxy, phenyloxy, halogen-lower alkoxy, such as trifluoromethoxy, 2,2,2-trifluoroethoxy or 1,1,2,2-tetrafluoroethoxy, or lower alkoxy which is substituted by mono- or bicyclic heteroaryl comprising one or two nitrogen atoms, preferably lower alkoxy which is substituted by imidazolyl, such as 1H-imidazol-1-yl, pyrrolyl, benzimidazolyl, such as 1-benzimidazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, indolyl or thiazolyl.

Esterified hydroxy is especially lower alkanoyloxy, benzoyloxy, lower alkoxycarbonyloxy, such as tert-butoxycarbonyloxy, or phenyl-lower alkoxycarbonyloxy, such as benzyloxycarbonyloxy.

Esterified carboxy is especially lower alkoxycarbonyl, such as tert-butoxycarbonyl, iso-propoxycarbonyl, methoxycarbonyl or ethoxycarbonyl, phenyl-lower alkoxycarbonyl, or phenyloxycarbonyl.

Alkanoyl is primarily alkylcarbonyl, especially lower alkanoyl, e.g. acetyl.

N-Mono- or N,N-disubstituted carbamoyl is especially substituted by one or two substituents independently selected from lower alkyl, phenyl-lower alkyl and hydroxy-lower alkyl, or lower alkylene, oxa-lower alkylene or aza-lower alkylene optionally substituted at the terminal nitrogen atom.

A mono- or bicyclic heteroaryl group comprising zero, one, two or three ring nitrogen atoms and zero or one oxygen atom and zero or one sulfur atom, which groups in each case are unsubstituted or mono- or polysubstituted, refers to a heterocyclic moiety that is unsaturated in the ring binding the heteroaryl radical to the rest of the molecule in formula IX and is preferably a ring, where in the binding ring, but optionally also in any annealed ring, at least one carbon atom is replaced by a heteroatom selected from the group consisting of nitrogen, oxygen and sulfur; where the binding ring preferably has 5 to 12, more preferably 5 or 6 ring atoms; and which may be unsubstituted or substituted by one or more, especially one or two, substituents selected from the group defined above as substituents for aryl, most preferably by lower alkyl, such as methyl, lower alkoxy, such as methoxy or ethoxy, or hydroxy. Preferably the mono- or bicyclic heteroaryl group is selected from 2H-pyrrolyl, pyrrolyl, imidazolyl, benzimidazolyl, pyrazolyl, indazolyl, purinyl, pyridyl, pyrazinyl, pyrimidinyl, pyridazinyl, 4H-quinolizinyl, isoquinolyl, quinolyl, phthalazinyl, naphthyridinyl, quinoxalyl, quinazolinyl, quinnolinyl, pteridinyl, indolizinyl, 3H-indolyl, indolyl, isoindolyl, oxazolyl, isoxazolyl, thiazolyl, isothiazolyl, triazolyl, tetrazolyl, furazanyl, benzo[d]pyrazolyl, thienyl and furanyl. More preferably the mono- or bicyclic heteroaryl group is selected from the group consisting of pyrrolyl, imidazolyl, such as 1H-imidazol-1-yl, benzimidazolyl, such as 1-benzimidazolyl, indazolyl, especially 5-indazolyl, pyridyl, especially 2-, 3- or 4-pyridyl, pyrimidinyl, especially 2-pyrimidinyl, pyrazinyl, isoquinolinyl, especially 3-isoquinolinyl, quinolinyl, especially 4- or 8-quinolinyl, indolyl, especially 3-indolyl, thiazolyl, benzo[d]pyrazolyl, thienyl, and furanyl. In one preferred embodiment of the invention the pyridyl radical is substituted by hydroxy in ortho position to the nitrogen atom and hence exists at least partially in the form of the corresponding tautomer which is pyridin-(1H)2-one. In another preferred embodiment, the pyrimidinyl radical is substituted by hydroxy both in position 2 and 4 and hence exists in several tautomeric forms, e.g. as pyrimidine-(1 H, 3H)2,4-dione.

Heterocyclyl is especially a five, six or seven-membered heterocyclic system with one or two heteroatoms selected from the group comprising nitrogen, oxygen, and sulfur, which may be unsaturated or wholly or partly saturated, and is unsubstituted or substituted especially by lower alkyl, such as methyl, phenyl-lower alkyl, such as benzyl, oxo, or heteroaryl, such as 2-piperazinyl; heterocyclyl is especially 2- or 3-pyrrolidinyl, 2-oxo-5-pyrrolidinyl, piperidinyl, N-benzyl-4-piperidinyl, N-lower alkyl-4-piperidinyl, N-lower alkyl-piperazinyl, morpholinyl, e.g. 2- or 3-morpholinyl, 2-oxo-1H-azepin-3-yl, 2-tetrahydrofuranyl, or 2-methyl-1,3-dioxolan-2-yl.

Salts are especially the pharmaceutically acceptable salts of compounds of formula IX.

Such salts are formed, for example, as acid addition salts, preferably with organic or inorganic acids, from compounds of formula IX with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, a compound of formula IX may also form internal salts.

For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

In view of the close relationship between the novel compounds in free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient.

Compounds within the scope of formula IX and the process for their manufacture are disclosed in WO 04/005281 published on Jan. 15, 2004 which is hereby incorporated into the present application by reference. A preferred compound is 4-Methyl-3-[[4-(3-pyridinyl)-2-pyrimidinyl]amino]-N-[5-(4-methyl-1H-imidazol-1-yl)-3-(trifluoromethyl)phenyl]benzamide, (compound X) or a pharmaceutically acceptable salt thereof.

The term “compounds decreasing the c-Src activity” as used herein includes, but is not limited to, compounds inhibiting the c-Src protein tyrosine kinase activity as defined above and to SH2 interaction inhibitors such as those disclosed in WO97/07131 and WO97/08193. Preferably, in the present invention compounds decreasing the c-Src activity are SH2 interaction inhibitors or, more preferably, compounds inhibiting the c-Src protein tyrosine kinase activity as defined above.

It will be understood that references to the pharmacologically active compounds mentioned herein are meant to also include the pharmaceutically acceptable salts. If compounds inhibiting the c-Src protein tyrosine kinase activity or a combination partner (a) or (b) have, for example, at least one basic center, they can form acid addition salts. The combination partners (a) and (b) having an acid group (for example COOH) can also form salts with bases. The pharmacologically active compounds mentioned herein may also be used in form of a hydrate or include other solvents used for crystallization.

Additionally, the present invention relates to a method of treating a warm-blooded animal having leukemia comprising administering to the animal at least one compound inhibiting the c-Src protein tyrosine kinase activity and the Bcr-Abl tyrosine kinase activity, in a quantity which is therapeutically effective against leukemia, in which method said compounds can also be present in the form of their pharmaceutically acceptable salts. Preferably, such compound is a compound of formula V.

A combination which comprises (a) at least one compound decreasing the c-Src activity and (b) pyrimidylaminobenzamide, wherein the active ingredients are present in each case in free form or in the form of a pharmaceutically acceptable salt, and optionally at least one pharmaceutically acceptable carrier will be referred to hereinafter as a COMBINATION OF THE INVENTION.

The nature of proliferative diseases like leukemia is multifactorial. Under certain circumstances, drugs with different mechanisms of action may be combined. However, just considering any combination of drugs having different mode of action does not necessarily lead to combinations with advantageous effects.

All the more surprising is the experimental finding that the administration of a COMBINATION OF THE INVENTION, results not only in a beneficial effect, especially a synergistic therapeutic effect, e.g. with regard to slowing down, arresting or reversing the progress of leukemia or a longer duration of drug response, but also in further surprising beneficial effects, e.g. less side-effects, an improved quality of life and a decreased mortality and morbidity, compared to a monotherapy applying only one of the pharmaceutically active ingredients used in the COMBINATION OF THE INVENTION.

A further benefit is that lower doses of the active ingredients of the COMBINATION OF THE INVENTION can be used, for example, that the dosages need not only often be smaller, but can be also applied less frequently, or can be used in order to diminish the incidence of side-effects. This is in accordance with the desires and requirements of the patients to be treated.

The utility of the COMBINATION OF THE INVENTION for the treatment of leukemia can be demonstrated, e.g., in the proliferation test using bcr-Abl transfected 32D cells as follows:

The proliferation test using bcr-Abl transfected 32D cells with a COMBINATION OF THE INVENTION is carried out as described above with the following changes. Two combination partners are mixed in fixed ratios. Threefold serial dilutions of this mixture or the combination partners alone are added to the cells seeded in 96 well tissue culture plates as described above. The effects on 32D-bcr-Abl cell proliferation of a COMBINATION OF THE INVENTION is evaluated and compared with the effects of the single combination partners using CalcuSyn, a dose-effect analyzer software for single and multiple drugs (distributed by Biosoft, Cambridge).

One particular benefit of the present invention is the fact that the leukemia that can be treated with a compound inhibiting the c-Src protein tyrosine kinase activity or with the COMBINATION OF THE INVENTION can be such leukemia which is resistant to monotherapy employing N-{5-[4-(4-methyl-piperazino-methyl)-benzoylamido]-2-methylphenyl}-4-(3-pyridyl)-2-pyrimidine-amine (STI571) as sole active agent, e.g. leukemia of such patients who initially had responded to STI571 and then relapsed. Very especially, compounds inhibiting the c-Src protein tyrosine kinase activity and COMBINATIONS OF THE INVENTION can be used for the treatment of patients in the advanced stage (blast crisis phase) of CML. STI571 can also be administered as marketed under the trademark GLIVEC™ or GLEEVEC™.

The person skilled in the pertinent art is fully enabled to select further relevant test models to prove the hereinbefore and hereinafter mentioned beneficial effects on leukemia of a COMBINATION OF THE INVENTION. The pharmacological activity of a COMBINATION OF THE INVENTION may, for example, be demonstrated in a suitable clinical study. Suitable clinical studies are, for example, open label non-randomized, dose escalation studies in patients with advanced leukemia. Such studies prove in particular the synergism observed with the COMBINATIONS OF THE INVENTION. The beneficial effects on leukemia can be determined directly through the results of these studies or by changes in the study design which are known as such to a person skilled in the art. For example, the combination partner (b) can be administered with a fixed dose and the dose of the combination partner (a) is escalated until the Maximum Tolerated Dosage is reached. Alternatively, a placebo-controlled, double blind study can be conducted in order to prove the benefits of the COMBINATION OF THE INVENTION mentioned herein.

In one embodiment of the invention, the compound inhibiting the c-Src protein tyrosine kinase activity is selected from pyrrolopyrimidines, especially pyrrolo[2,3-d]pyrimidines, purines, pyrazopyrimidines, especially pyrazo[3,4-d]pyrimidines, pyrazopyrimidines, especially pyrazo[3,4-d]pyrimidines and pyridopyrimidines, especially pyrido[2,3-d]-pyrimidines. Particularly preferred are the compounds of formula I, II, Ill, IV, V, VI, VII and VIII, especially the compound of formula I and formula V.

Especially preferred is a combination comprising a compound of formula I and compound X or the pharmaceutically acceptable salts thereof. Furthermore, especially preferred is a combination comprising a compound of formula V and compound X or the pharmaceutically acceptable salts thereof.

The invention pertains also to the use of the COMBINATION OF THE INVENTION for the treatment of leukemia and for the preparation of a medicament for the treatment of leukemia.

The COMBINATION OF THE INVENTION can be a combined preparation or a pharmaceutical composition.

The term “a combined preparation”, as used herein defines especially a “kit of parts” in the sense that the combination partners (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b). The ratio of the total amounts of the combination partner (a) to the combination partner (b) to be administered in the combined preparation can be varied, e.g. in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners (a) and (b), in particular a synergism, e.g. a more than additive effect, additional advantageous effects, less side effects, a combined therapeutical effect in a non-effective dosage of one or both of the combination partners (a) and (b), and very preferably a strong synergism of the combination partners (a) and (b).

It is one objective of this invention to provide a pharmaceutical composition comprising a quantity, which is jointly therapeutically effective against leukemia comprising the COMBINATION OF THE INVENTION. In this composition, the combination partners (a) and (b) can be administered together, one after the other or separately in one combined unit dosage form or in two separate unit dosage forms. The unit dosage form may also be a fixed combination.

The pharmaceutical compositions for separate administration of the combination partners (a) and (b) and for the administration in a fixed combination, i.e. a single galenical compositions comprising at least two combination partners (a) and (b), according to the invention can be prepared in a manner known per se and are those suitable for enteral, such as oral or rectal, and parenteral administration to mammals (warm-blooded animals), including man, comprising a therapeutically effective amount of at least one pharmacologically active combination partner alone or in combination with one or more pharmaceutically acceptable carries, especially suitable for enteral or parenteral application.

Novel pharmaceutical composition contain, for example, from about 10% to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each of the combination partner of the COMBINATION OF THE INVENTION may be administered simultaneously or sequentially and in any order, and the components may be administered separately or as a fixed combination. For example, the method of treatment of leukemia according to the present invention may comprise (i) administration of the combination partner (a) in free or pharmaceutically acceptable salt form and (ii) administration of a combination partner (b) in free or pharmaceutically acceptable salt form, simultaneously or sequentially in any order, in jointly therapeutically effective amounts, preferably in synergistically effective amounts, e.g. in daily dosages corresponding to the amounts described herein. The individual combination partners of the COMBINATION OF THE INVENTION can be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimes of simultaneous or alternating treatment and the term “administering” is to be interpreted accordingly.

The effective dosage of each of the combination partners employed in the COMBINATION OF THE INVENTION may vary depending on the particular compound or pharmaceutical composition employed, the mode of administration, the condition being treated, the severity of the condition being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

The compound of formula IX can be administered by any route including orally, parenterally, e.g., intraperitoneally, intravenously, intramuscularly, subcutaneously, intratumorally, or rectally, or enterally. Preferably the compound of formula I is administered orally, preferably at a daily dosage of 1-300 mg/kg body weight or, for most larger primates, a daily dosage of 50-5000, preferably 500-3000 mg. A preferred oral daily dosage is 1-75 mg/kg body weight or, for most larger primates, a daily dosage of 10-2000 mg, administered as a single dose or divided into multiple doses, such as twice daily dosing.

Compounds inhibiting the c-Src protein tyrosine kinase activity, e.g. the compound of formula I, is preferably administered orally to a human in a dosage in the range of about 100 to 2000 mg/day, more preferably 500 to 1500 mg/day, e.g. 1000 mg/day.

If BAY 43-9006 is employed as a combination partner, it is preferably administered orally at doses of up to 800 mg twice daily.

Moreover, the present invention provides a commercial package comprising as active ingredients COMBINATION OF THE INVENTION, together with instructions for simultaneous, separate or sequential use thereof in the treatment of leukemia.

The present invention pertains to:
1. A combination which comprises (a) at least one compound decreasing the c-Src activity protein tyrosine kinase and (b) a pyrimidylaminobenzamide compound of formula IX, wherein the active ingredients are present in each case in free form or in the form of a pharmaceutically acceptable salt, and optionally at least one pharmaceutically acceptable carrier; for simultaneous, separate or sequential use.
2. the combination under 1 wherein compound inhibiting the c-Src protein tyrosine kinase activity is selected from a compound of formula I and a compound of formula V.
3. the combination under 1 or 2 wherein compound (b) is 4-Methyl-3-[[4-(3-pyridinyl)-2-pyrimidinyl]amino]-N-[5-(4-methyl-1H-imidazol-1-yl)-3-(trifluoromethyl)phenyl]benzamide, compound X.
4. the combination as mentioned above for use in the therapeutic or diagnostic treatment of the animal or human body.
5. Use of the combination under 1 to 3 for the manufacture of a medicament for the treatment of leukemia.
6. Method of treating a warm-blooded animal having leukemia comprising administering to the animal (a) at least one compound decreasing the c-Src activity and (b) a pyrimidylaminobenzamide compound of formula (IX) in a quantity which is jointly therapeutically effective against leukemia.
7. Method under 6, wherein the compound inhibiting the c-Src protein tyrosine kinase activity is selected from a compound of formula I and a compound of formula V.
8. Method under 6 or 7 wherein said leukemia is resistant to monotherapy employing N-{5-[4-(4-methyl-piperazino-methyl)-benzoylamido]-2-methylphenyl}-4-(3-pyridyl)-2-pyrimidine-amine as sole active agent.
9. Method under 6 or 7 wherein said leukemia is chronic myelogenous leukemia.
10. A pharmaceutical composition comprising a quantity which is jointly therapeutically effective against leukemia of a combination according to any one of claims 1 to 3 and at least one pharmaceutically acceptable carrier.
11. A commercial package comprising at least one c-Src protein tyrosine kinase activity inhibitor together with instructions for use thereof in the treatment of leukemia.
12. A commercial package comprising (a) at least one compound decreasing the c-Src protein tyrosine kinase activity and (b) a pyrimidylaminobenzamide compound of formula (IX), together with instructions for simultaneous, separate or sequential use thereof in the treatment of leukemia.
13. A combination under 1 wherein the src inhibitor is selected from bosutinib and dasatinib.

1. Combination which comprises (a) at least one compound decreasing the c-Src activity protein tyrosine kinase and (b) a pyrimidylaminobenzamide compound of formula IX, wherein the active ingredients are present in each case in free form or in the form of a pharmaceutically acceptable salt, and optionally at least one pharmaceutically acceptable carrier; for simultaneous, separate or sequential use.

2. Combination according to claim 1 wherein compound inhibiting the c-Src protein tyrosine kinase activity is selected from a compound of formula I 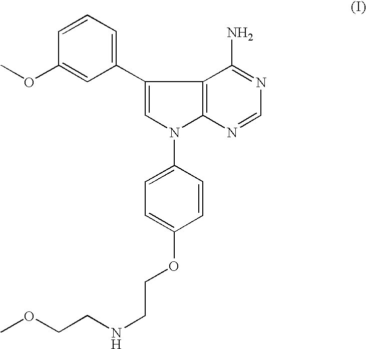
and a compound of formula V

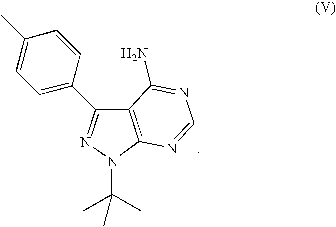

3. Combination according to claim 1 wherein compound (b) is 4-Methyl-3-[[4-(3-pyridinyl)-2-pyrimidinyl]amino]-N-[5-(4-methyl-1H-imidazol-1-yl)-3-(trifluoromethyl)phenyl]benzamide, compound X.

4. A combination according to claim 1 for use in the therapeutic or diagnostic treatment of the animal or human body.

5. (canceled)

6. Method of treating a warm-blooded animal having leukemia comprising administering to the animal (a) at least one compound decreasing the c-Src activity and (b) a pyrimidylaminobenzamide compound of formula (IX) in a quantity which is jointly therapeutically effective against leukemia.

7. Method according to claim 6, wherein the compound inhibiting the c-Src protein tyrosine kinase activity is selected from a compound of formula I 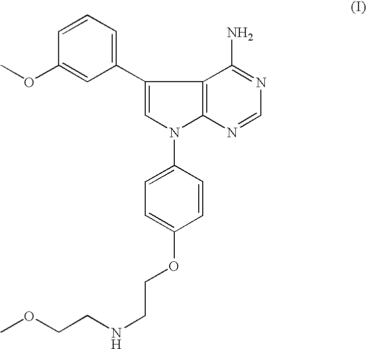
and a compound of formula V

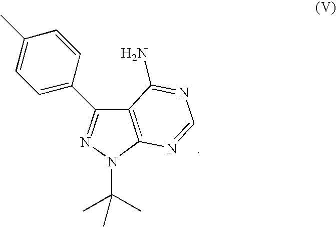

8. Method according to claim 6 wherein said leukemia is resistant to monotherapy employing N-{5-[4-(4-methyl-piperazino-methyl)-benzoylamido]-2-methylphenyl}-4-(3-pyridyl)-2-pyrimidine-amine as sole active agent.

9. Method according to claim 6 wherein said leukemia is chronic myelogenous leukemia.

10. A pharmaceutical composition comprising a quantity which is jointly therapeutically effective against leukemia of a combination according to claim 1 and at least one pharmaceutically acceptable carrier.

11. A commercial package comprising at least one c-Src protein tyrosine kinase activity inhibitor together with instructions for use thereof in the treatment of leukemia.

12. A commercial package comprising (a) at least one compound decreasing the c-Src protein tyrosine kinase activity and (b) a pyrimidylaminobenzamide compound of formula (IX), together with instructions for simultaneous, separate or sequential use thereof in the treatment of leukemia.

13. A combination according to claim 1 wherein the src inhibitor is selected from bosutinib and dasatinib.
